(19) United States
(12) Patent Application Publication
(10) Pub. No.: US 2024/0261537 A1
(43) Pub. Date: Aug. 8, 2024

(54) RADIAL DEPLOYMENT CATHETER

(71) Applicant: ClearPoint Neuro, Inc., Solana Beach, CA (US)

(72) Inventors: Nathan Williams, San Diego, CA (US); Ernesto Salegio, Solana Beach, CA (US)

(21) Appl. No.: 18/430,073

(22) Filed: Feb. 1, 2024

Related U.S. Application Data
(60) Provisional application No. 63/483,606, filed on Feb. 7, 2023.
(60) Provisional application No. 63/482,885, filed on Feb. 2, 2023.

(51) Int. Cl. A61M 25/00; A61M 25/06

(52) U.S. Cl. CPC A61M 25/0023; A61M 25/0662; A61M 2025/0024; A61M 2025/0681

(57) ABSTRACT

A cannula and catheter system includes a cannula or catheter formed of an elongated body having a central lumen. The catheter has an outer sheath of rigid material. An inner tube is co-axially positioned within the outer sheath wherein the inner tube is made of a material that is less stiff than the outer sheath or wherein the outer sheath is more stiff or rigid than one or more sheaths, tubes or cannulas slidably nested within the outer sheath.

### CROSS REFERENCE TO RELATED APPLICATIONS

The present application claims priority to U.S. Provisional Application No. 63/483,606, filed on Feb. 7, 2023, and entitled “RADIAL DEPLOYMENT CATHETER,” and U.S. Provisional Application No. 63/482,885, filed on Feb. 2, 2023, and entitled “RADIAL DEPLOYMENT CATHETER” the entirety of each of which are incorporated by reference herein.

### BACKGROUND

To perform direct intraparenchymal drug delivery in the brain and spinal cord, cannula devices are guided through gray and white anatomical tissue to reach specific anatomical regions at pre-determined depths. Current devices are limited in usability due to the rigid outer components (such as ceramic and fused silica) of the cannula, which limits the shape of the cannula to a fixed trajectory. As a result, advancement of the cannula through the anatomy can only be performed along a single linear trajectory. Such an arrangement does not allow for advancement in angular trajectories once the main body of the cannula has been inserted into the brain and spinal cord parenchyma.

This presents a problem because many anatomical regions in the brain and spinal cord have distinct shapes and angles. Currently, successful delivery of therapeutics to such target locations thus requires multiple cannula insertions to allow for maximum therapeutic distribution through each tissue. A solution to overcome such limitations is described herein.

### SUMMARY

Disclosed is a cannula and catheter system that is configured to overcome the aforementioned drawbacks. The system includes a cannula or catheter formed of an elongated body having a central lumen. The catheter has an outer sheath of rigid material. An inner tube is co-axially positioned within the outer sheath wherein the inner tube is made of a material that is less stiff than the outer sheath or wherein the outer sheath is more stiff or rigid than one or more sheaths, tubes or cannulas slidably nested within the outer sheath. The inner tube (or tubes) is shape set to form any of a wide variety of shapes or contours when the inner tube is unimpeded such as when the inner tube is not positioned inside the outer sheath. The shape or contour can be straight, curved, curvilinear or any other shape or contour. An innermost member (such as an inner tube) having a central lumen is co-axially positioned inside the inner tube. The outer sheath, inner tube, and innermost member are telescopically positioned relative to one another such that they can be movably positioned relative to one another while remaining co-axial.

A control system is coupled to the catheter. The control system includes one or more mechanisms to control an orientation/angle and extension/retraction of components of the catheter 105, such as the shape set inner tube and the innermost member. The control system enables a user to achieve catheter positions and trajectories through tissue that cannot be achieved in a conventional system.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a distal region of a catheter wherein components of the catheter are in an extended state.

FIG. 2 shows the distal region of the catheter with the components of the catheter in a retracted state.

FIGS. 3 and 4 show the distal region of the catheter with components of the catheter in a partially extended state.

FIG. 5 shows the catheter having a control element coupled to a proximal region of the catheter.

FIG. 6 shows the control element with some components translucent to show internal features.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

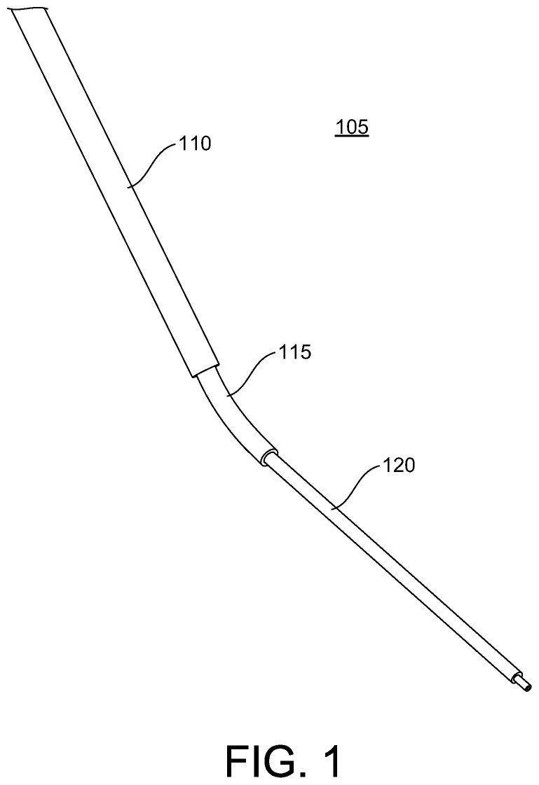

FIG. 1 shows a distal region of a catheter 105 that is formed of an elongated body having at least one axial lumen that forms at least one distal opening and at least one proximal opening. In a non-limiting embodiment, the catheter 105 is configured to be inserted into a brain region of a patient such as via a transcranial approach pathway.

The catheter 105 is formed of a rigid outer sheath 110, a shape set inner tube 115, and an innermost member 120 that forms a central lumen. The outer sheath 110, inner tube, 115, and innermost member 120 are configured to be telescopically moved relative to one another along a long axis of the catheter 105. That is, the inner tube, 115 and innermost member 120 can co-axially slide relative to one another and relative to the outer sheath 110. FIG. 1 shows the catheter 105 in an extended state such that the innermost member 120 is slid to a position projects distally outward from the inner tube 115 and the inner tube 115 is slid to a position that projects distally outward from the outer sheath 110. The components of the catheter 105 (outer sheath, inner tube 115, and innermost member 120) can be removably attachable such that the device can be pre-assembled or assembled by a user prior to use. The components can also be manufactured of any of a wide variety of materials including plastic and/or metal (including Nitinol.) The entire length of any of the components can be made of a unitary material or different materials along their lengths. The catheter can be used for various purposes including, for example, the delivery of therapeutics ranging from gene therapies, cells therapies, etc.

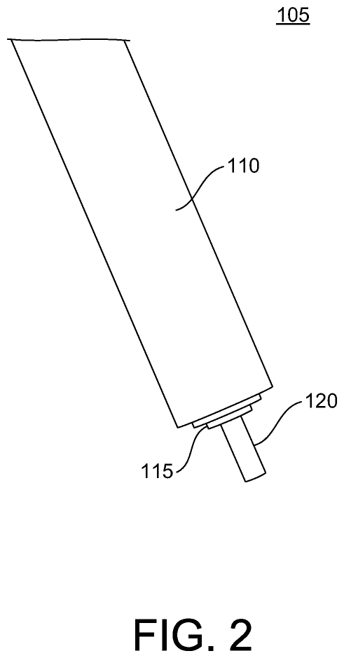

FIG. 2 shows the distal region of the catheter 105 in a state, which can be a default or initial state, wherein the inner tube 115 and the innermost member 120 are fully retracted into the outer sheath 110. Thus, the distal ends of the inner tube 115 and the innermost member are flush or substantially flush with a distal end of the outer sheath 110. The inner tube 115 and the innermost member 120 are more flexible relative to a flexibility of the outer sheath 110 such that they both conform to the shape (such as a straight shape or other contoured shape which is not necessarily straight) of the more rigid outer sheath 110 while retracted inside the outer sheath 110. In this state, the catheter 105 can be inserted into the anatomy along a straight line (as defined by the outer sheath 110) or along another predetermined trajectory of a desired shape/contour such as until the distal tip of the catheter 105 is located at a predetermined location within the anatomy. The rigid outer sheath 110 can then be fixed in place. The catheter 105 can also be oriented or rotated to a predetermined orientation relative to the anatomy.

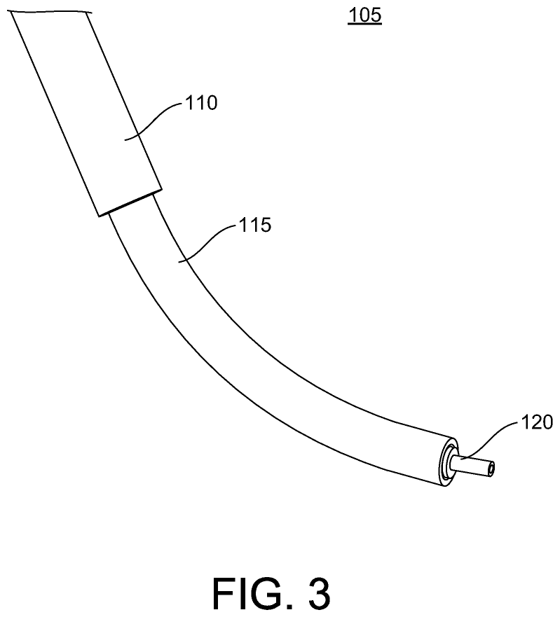

FIG. 3 shows the catheter 105 wherein the inner tube 115 (which has a default or predetermined straight or curved shape when unimpeded by the outer sheath 110) is extended distally outward of the distal tip of the outer sheath 110 by a desired length. As the shape set inner tube extends beyond distal tip of the rigid outer sheath 110 as shown in FIG. 3, the outer sheath 110 no longer constrains the shape of the inner tube 115 such that the inner tube 115 assumes its shape set configuration. The shape set is the shape or contour of the inner tube (or a portion thereof) when unimpeded by being nested inside the outer sheath. The shape set configuration can include at least one bend for example. The predetermined shape set will not revert to a prior shape or assume a different shape once inside of the brain or spinal cord in non-limiting example. It should be appreciated that the unimpeded, shape set configuration of the inner tube can be curved, straight, or curvilinear.

The innermost member 120, which is nested inside the inner tube 110, extends in conjunction with the shape set inner tube 115 such that the innermost member 120 remains at least partially nested within the inner tube 115 as the inner tube is extended outward relative to the outer sheath. The catheter 105 can be orientated to extend the distal end of the catheter 105 along a desired trajectory or desired direction wherein a final bend angle is a function of at least the arc length and bend radius of the various components of the catheter 105. Therefore, the final angle of trajectory can be modified by controlling how far the shape set inner tube 115 is extended beyond the rigid outer sheath 110.

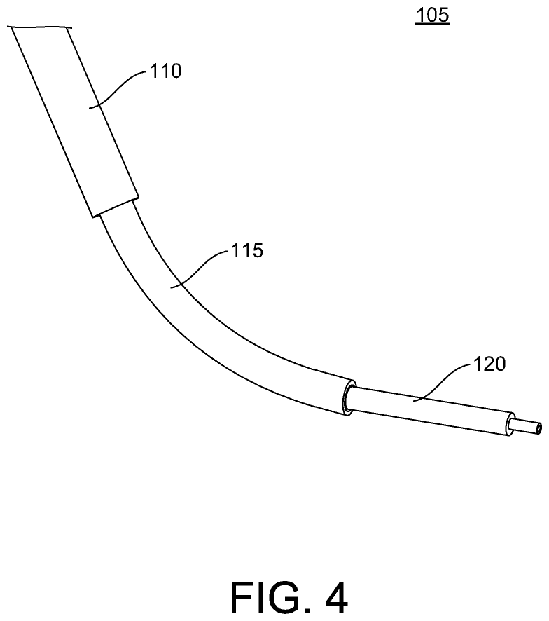

FIG. 4 shows a subsequent configuration of the catheter 105 wherein a distal end of the innermost member 120 is extended beyond the distal tip of the shape set inner tube 115. Once a portion of the innermost member 120 exits the distal end of the shape set inner tube 115, that portion continues in a straight or curvilinear line along an exit trajectory defined by an endmost trajectory of the inner tube 115. The innermost member 120 can be extended to multiple positions along this pathway. The innermost member 120 can then be fully retracted into the inner tube 115. If desired, a user can then extend/retract the inner tube 115 to a new angle or trajectory having a desired exit trajectory. If the shape set inner tube 115 is fully retracted, then the rigid outer sheath 110 and/or the inner tube 115 can then be adjusted (such as via rotation) to a new angle and can then fully retracted into the inner tube. The shape set inner tube can then be extended/retracted to a new angle. If the shape set inner tube 120 is fully retracted, then the rigid outer sheath can then be adjusted to a new angle and/or depth or be removed to complete a treatment procedure for the anatomy.

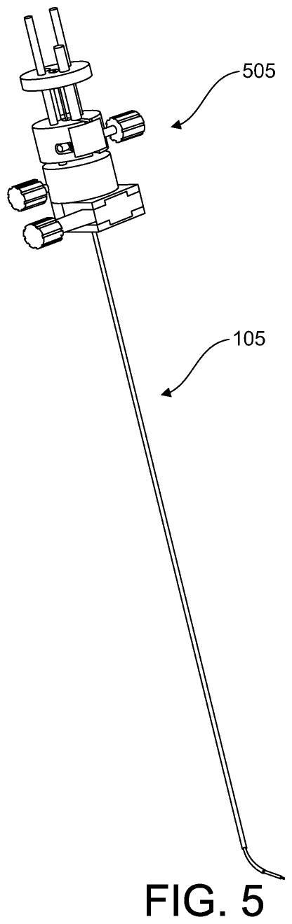

The catheter 105 can be used pursuant to a control system that includes mechanisms to control orientation/angle and extension/retraction of components of the catheter 105, such as the shape set inner tube 115 and the innermost member 120 relative to one another. FIG. 5 shows an example embodiment of such a control system 505 that is coupled to a proximal region of the catheter 105. The control system 505 includes one or more actuators, such as knobs, buttons, wheels, etc., that can be used to control relative position and orientation of the catheter 105 and its components.

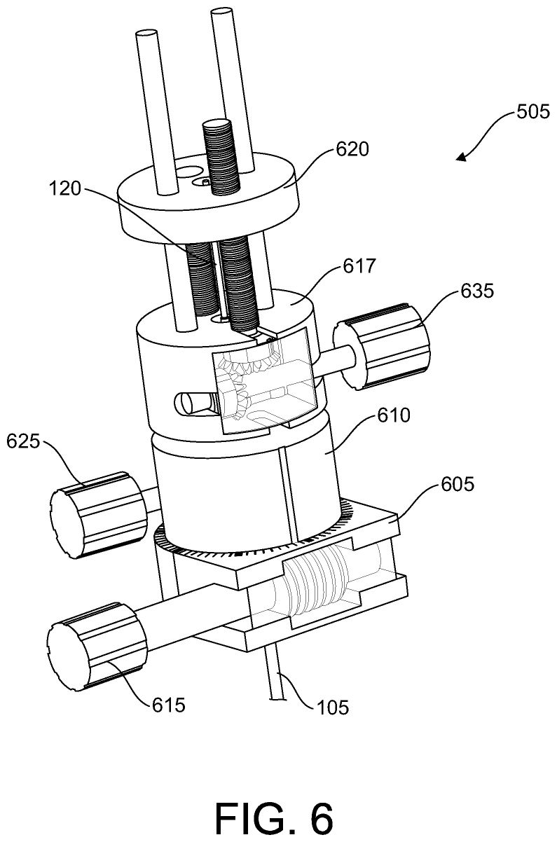

FIG. 6 shows the control system 505 with some components translucent to show internal features. The control system includes a dock 605 comprising a structure that forms a stage, such as a flat surface that is coupled to a base 610. In an embodiment, the base 610 is a cylindrical body positioned atop the dock 605. The control system can include one or more mechanical or electromechanical components such as knobs, gears and other features that can be actuated to control the catheter 105 such as the movement and orientation of the outer sheath 110, shape set inner tube 115, and innermost member 120 relative to one another. In an embodiment, each of the outer sheath 110, shape set inner tube 115, and innermost member 120 has at least one corresponding control element, such as knobs, gears and other features, that control position and rotation of the respective component of the catheter 105.

In an example embodiment, the base 610 is mechanically coupled to the dock 605 such that turning a knob 615 causes the base 610 to rotate about an axis coaxially aligned with the catheter 105. The mechanical coupling can include a threaded engagement between a structure attached to the knob 615 and the base 610 for example although this can vary. The mechanism makes it possible to adjust the rotational orientation (i.e., rotation about a long axis of the inner tube) of the shape set inner tube of the catheter 105.

With reference still to FIG. 6, the control system 505 includes an angle stage 617 that floats above the base 610 and that is connected thereto via risers (such as columns) that maintain alignment with the base 610. The columns can extend through the angle stage 617 and the base 610. The risers can move up and down relative to the base 610. An innermost member stage 620 is connected in the same manner to the base 610 or angle stage 617.

An actuator, such as a knob and/or lead screw 625, can be actuated to control a distance between the angle stage 617 and the base 610. The lead screw 625 is coupled to gears and knobs such that turning (or otherwise actuating) the lead screw 625 causes the angle stage 617 to move up or down relative to the base 610. The shape set inner tube 115 is connected to the angle stage 617 such that actuating the lead screw 625 causes the shape set inner tube 115 to slide relative to the outer sheath such as to retract or extend from the rigid outer sheath 110. The stages may include features that make them inherently stable when not manually raised/lowered, such as work gears or lead screws. The stages may also include locking mechanisms to ensure they do not move unintentionally. The device can include markings to indicate the distance (or corresponding angle) that the shape set inner tube is extended.

The control system further includes a knob and/or lead screw 635 configured to control a distance between the lumen stage 620 and the angle stage 617. In an embodiment, both stages 615 and 620 tend to move up and down in unison relative to the base. However, using gears and knobs to turn the lead screw 635 causes the lumen stage 620 to move up or down relative to the angle stage 617. The innermost member 120 is connected to stage 620 such that actuation of the lead screw 635 causes the innermost member 120 to retract or extend from the shape set inner tube 115. The device can include markings to indicate the distance that the inner lumen member is extended.

In use, the catheter 105 is deployed into anatomy toward a target location, which can be in a brain or spinal cord for example although the device is not limited to use within the brain only. For example, the device can be used in the spinal cord as well as other anatomic regions. A user can actuate the various components of the control system 505 to control trajectory and location of the catheter by manipulating the relative positions of outer sheath 110, inner tube 115, and innermost member 120.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A catheter system, comprising:
an elongated outer sheath having a first stiffness;
an elongated inner tube co-axially contained within the outer sheath, wherein the inner tube has a pre-set shape when not restrained by the outer sheath and wherein a shape of the inner tube conforms to a shape of the outer sheath when the inner tube is contained within the outer sheath;
an elongated innermost member co-axially contained within the inner tube;
wherein the inner tube and the innermost member are telescopically moveable relative to one another and the outer sheath;
a control element coupled to the outer sheath, the inner tube, and the innermost member, wherein the control element can be actuated to control relative movement of the outer sheath, the inner tube, and the innermost member.

2. The catheter system of claim 1, wherein the pre-set shape of the inner tube is curved.

3. The catheter system of claim 1, wherein the pre-set shape of the inner tube is straight.

4. The catheter system of claim 1, wherein the control element can be actuated to cause the inner tube to rotate relative to at least the outer sheath.

5. The catheter system of claim 1, wherein the control element can be actuated to cause the inner tube to extend or retract relative to the outer sheath.

6. The catheter system of claim 1, wherein the control element can be actuated to cause the inner tube to lock relative to the outer sheath.

7. The catheter system of claim 1, wherein the control element can be actuated to cause the innermost member to extend or retract relative to the outer sheath or inner tube.

8. The catheter system of claim 1, wherein the control element includes markings to indicate an extension or orientation of the inner tube relative to the outer sheath.

9. The catheter system of claim 1, wherein the control element includes markings to indicate an extension or orientation of the innermost member relative to the inner tube.

10. A catheter system, comprising:
an elongated outer sheath having a first stiffness;
an elongated inner tube co-axially contained within the outer sheath, wherein the inner tube has a pre-set shape when not restrained by the outer sheath and wherein a shape of the inner tube conforms to a shape of the outer sheath when the inner tube is contained within the outer sheath;
wherein the inner tube is telescopically moveable relative to one another and the outer sheath;
a control element coupled to the outer sheath and the inner tube, wherein the control element can be actuated to control relative movement of the outer sheath and the inner tube.

11. The catheter system of claim 10, wherein the pre-set shape of the inner tube is curved.

12. The catheter system of claim 10, wherein the pre-set shape of the inner tube is straight.

13. The catheter system of claim 10, wherein the control element can be actuated to cause the inner tube to rotate relative to at least the outer sheath.

14. The catheter system of claim 10, wherein the control element can be actuated to cause the inner tube to extend or retract relative to the outer sheath.

15. The catheter system of claim 10, wherein the control element can be actuated to cause the inner tube to lock relative to the outer sheath.

16. The catheter system of claim 10, further comprising an innermost member co-axially contained within the inner tube and wherein the control element can be actuated to cause the innermost member to extend or retract relative to the outer sheath or inner tube.

17. The catheter system of claim 10, wherein the control element includes markings to indicate an extension or orientation of the inner tube relative to the outer sheath.

18. The catheter system of claim 16, wherein the control element includes markings to indicate an extension or orientation of the innermost member relative to the inner tube.
